(19) United States
(12) Patent Application Publication
(10) Pub. No.: US 2004/0193265 A1
(43) Pub. Date: Sep. 30, 2004

(54) Spring-ball valve for voice prosthesis

(76) Inventors: Edmund V. Seder, Santa Barbara, CA (US); Mitchell A. Solis, Cunning, GA (US)

Correspondence Address: Marvin E Jacobs, Koppel Jacobs Patrick & Heybl, Suite 215, 2151 Alessandro Drive, Ventura, CA, 93001, US

(21) Appl. No.: 10/487,613

(22) PCT Filed: Dec. 20, 2002
(86) PCT No.: PCT/US02/41275

Related U.S. Application Data
(60) Provisional application No. 60/344,591, filed on Dec. 28, 2001.

(51) Int. Cl. A61F 2/20

(52) U.S. Cl. 623/9; 251/368; 137/512.3

(57) ABSTRACT

A voice prosthesis that is formed of a soft, elastomeric body containing a spring loaded ball valve, preferably a non-impervious, smooth ball such as a ruby ball biased by a flat helical spring against a seat mounted in a rigid cartridge. An insertion device includes a handle with expandable tip which enters and locks within the cartridge.

### TECHNICAL FIELD

The present invention relates to a voice prosthesis and prosthesis insertion device.

### BACKGROUND OF THE INVENTION

Soft prosthesis having soft silicone or polyurethane bodies can be invaded by growth of microorganisms into the body of the prosthesis when placed in body cavities such as the trachea. This is the typical failure mode for extended dwell voice prosthesis devices. Use of antimicrobial agents in devices having contact with tissue has not been completely successful due to irritation to the adjacent tissue.

### STATEMENT OF THE INVENTION

This invention relates to a soft polymer voice prosthesis incorporating a spring loaded ball valve, preferably a flat spring that compresses into a flat disc form such as straight or curved tabs raised from the surface of the disc such as a double helical spring loaded ruby ball valve and a device for inserting the prosthesis in a trachea-esophageal fistula.

The spring loaded ball valve is disposed in a hollow, rigid cartridge having a proximal end and a distal end. The open spring has a first end mounted at the distal end of the cartridge and has a second end within the cartridge for mounting the ball. The spring biases the ball toward the proximal end of the cartridge and is compressible toward the distal end of the cartridge on application of low pressure to the proximal surface of the ball. The proximal end of the cartridge has a diameter smaller than the first end and sealingly received the ball. An open end cap can be received on the distal end of the cartridge and can contain means for mounting the first end of the spring. The mounting means can include locking pins for locking the first end of the spring. The spring can be a helical spring formed of a spiral of flat material that compresses into a flat configuration. The spring can have a multiple helical configuration such as a double helical configuration.

The ball has a smooth surface impervious to microbial growth such as a ceramic material, suitably ruby, the spring is formed of a metal and the soft body is formed of polyurethane.

The proximal portion of the ball has a circular cross-section such as a spherical, conical or a segment thereof.

A voice prosthesis includes a tubular, hollow, elastomeric body having a central channel terminating in a proximal end and a distal end, the ball valve being fixedly received in the channel. The voice prosthesis can further include a particle flap hingedly mounted at the distal end of the body. The proximal end of the body can contain a hood extending axially from the proximal end of the cartridge. The hood can contain a groove for locking engagement with the end of an insertion device.

The voice prosthesis insertion device for inserting the voice prosthesis into a trachea-esophageal fistula comprises an elongated handle to be gripped by the hand of the user. The handle has a distal tip having a diameter such that it is slidingly received into the proximal end of the rigid cartridge. Means are present for expanding the distal end of the insertion tool to lock the end in place in the cartridge. Activation means on said handle activates the expansion means. The distal end of the handle can be slotted and can contain a protrusion which on expansion of the distal end engages and locks into a groove in the hood.

The voice prosthesis of the invention can be formed of unique materials for the valve and spring that do not allow microbial colonization of the working components of the device and therefore will extend the working life of the device. The use of a spring-ball valve in the prosthesis creates a device operating with parameters (pressure vs. flow, opening and closing pressures), which will remain constant with time, temperature and/or usage. The performance of a metal (such as stainless steel or titanium) plastic or composite material spring, is more repeatable than that of an elastomer-type device which vary in their elastic properties from batch to batch as well as within a batch. Use of thermoplastic materials for the cartridge and the end cap housing the spring permits use of ultrasonic welding for bonding, while simultaneously capturing the spring. The voice prosthesis of the invention is believed to be the first device that has identical opening and closing pressures. A voice prosthesis with a soft body formed of polyurethane has all the above advantages and the additional advantage of not allowing microbial colonization of any component of the device.

These and many other attendant advantages and attendant features of the invention will become apparent as the invention is disclosed in relation to the accompanying drawings.

### BRIEF DESCRIPTION OF THE DRAWINGS

FIG. 1 is a cross sectional view of a voice prosthesis in accordance with the invention;

FIG. 2 is a perspective view partially in section of another embodiment of the voice prosthesis of this invention;

FIG. 3 is a view in section taken along line 3-3 of FIG. 2.

FIG. 4 is a view in section of a first embodiment of an insertion device inserted into the proximal end of a voice prosthesis; and

FIG. 5 is a view partially in section of a second embodiment of an insertion device;

FIG. 6 is a view in section of an insertion device shown engaged with a voice prosthesis with the activation lever shown in un-locked position.

### DETAILED DESCRIPTION OF THE INVENTION

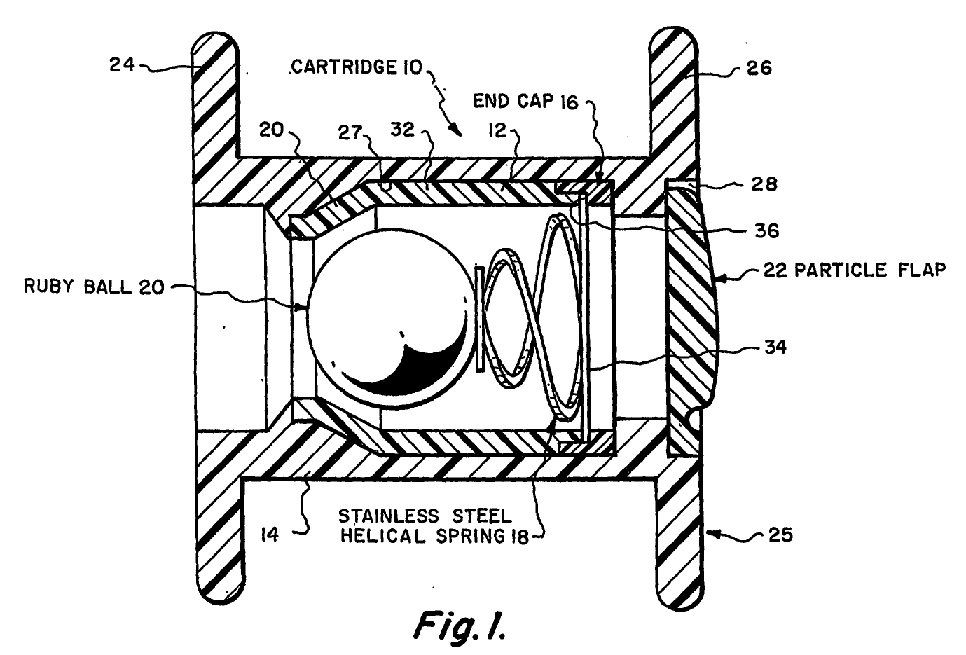

Referring now to FIG. 1, the voice prosthesis 10 of this invention includes a cartridge 12, a soft body 14, an end cap 16 for the cartridge, a helical spring 18 and a ball 20. A particle flap 22 may be mounted at the distal end 25 of the body 14. The body 14 has a hollow cylindrical section 27 containing cylindrical flanges 24, 26 mounted at each end of the section. The distal end 25 of the body 14 can have a recess or hood 28 in which the flap 22 is mounted.

The cartridge 12 and the end cap 16 can be formed of a biocompatible rigid, composite material, metal or engineering plastic. The cartridge has a front conical section 30 in which the ball 20 is seated and a cylindrical portion 32. The ends 34 of the spring 18 can be mounted in a recess 36 in the inside of the cap 16.

The conical section of the cartridge is difficult to mold. It can be replaced with a seat for the ball formed of metal or a hard metal oxide such as ruby or sapphire. Ruby and sapphire parts with conical or spherical seats are available from General Ruby and Sapphire Company.

The ball 20 is a solid body having a circular cross-section and a surface increasing in diameter from a first end to a second end such as a sphere, a cone or a segment thereof. The surface is impervious to biological growth and is preferably smooth and hard. The ball can be formed of a material such as, a metal oxide, a resin or a fiber or powder reinforced resin composite.

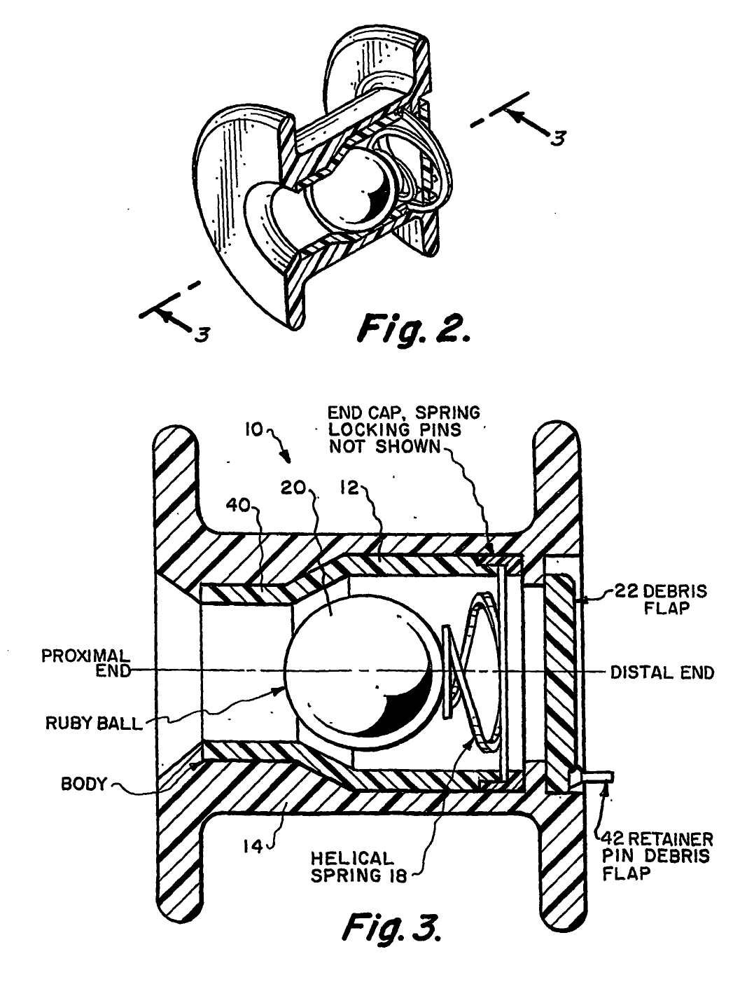

FIGS. 2 and 3 illustrate a voice prosthesis device 10 in which the cartridge 12 has an extended hood 40 at the proximal end of the device 10. The debris flap 22 is connected to the'soft body 14 by means of a retainer pin(s) with or without adhesive 42.

Pins in the cartridge body (cartridge retainer pins) can fit into notches located in the outer ring of the helical spring. These pins will locate the spring or springs to facilitate faster and more precise location during assembly. The cartridge retainer pins can also be used to stake the spring if ultrasonic welding of the cartridge body to end cap is performed. The cartridge retainer pins would positively locate the springs in place such that only breaking of the spring or pins would allow the spring to become separated from the device. The cartridge retainer pins would also allow multiple springs to be placed in the device such that their orientation would prevent coil binding and present a similar air path profile and pressure drop to that of a single spring. Stacking springs will allow such benefits as a greater spring rate at any giving spring travel or preload. By having the outer ring of the spring captured within the walls of the cartridge and cartridge end cap it is kept out of the air path for lower pressure drop and therefore better performance.

The helical spring design allows the spring to compress and extend uniformly, without drifting off axis. This along with the flat platform at the small end of the spring allows the ball to find the center of the cone for proper sealing.

A silicone or polyurethane flap on the exhaust side of the device can be used to prevent particles or decrease the amount of particles from entering the device. This flap would not be used as an air seal and would be extremely flexible to limit air resistance. A non-sealing flap of this type has not been used in prior voice prosthesis devices.

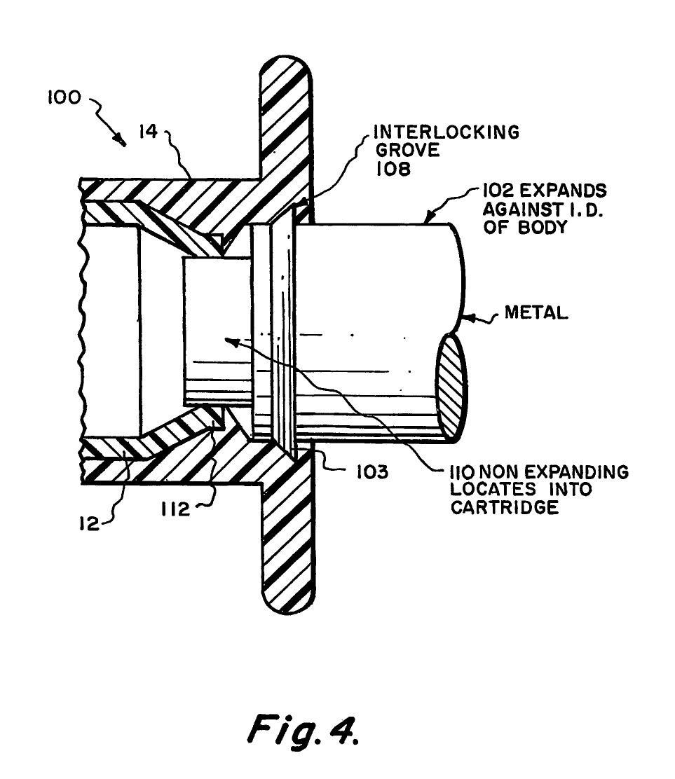

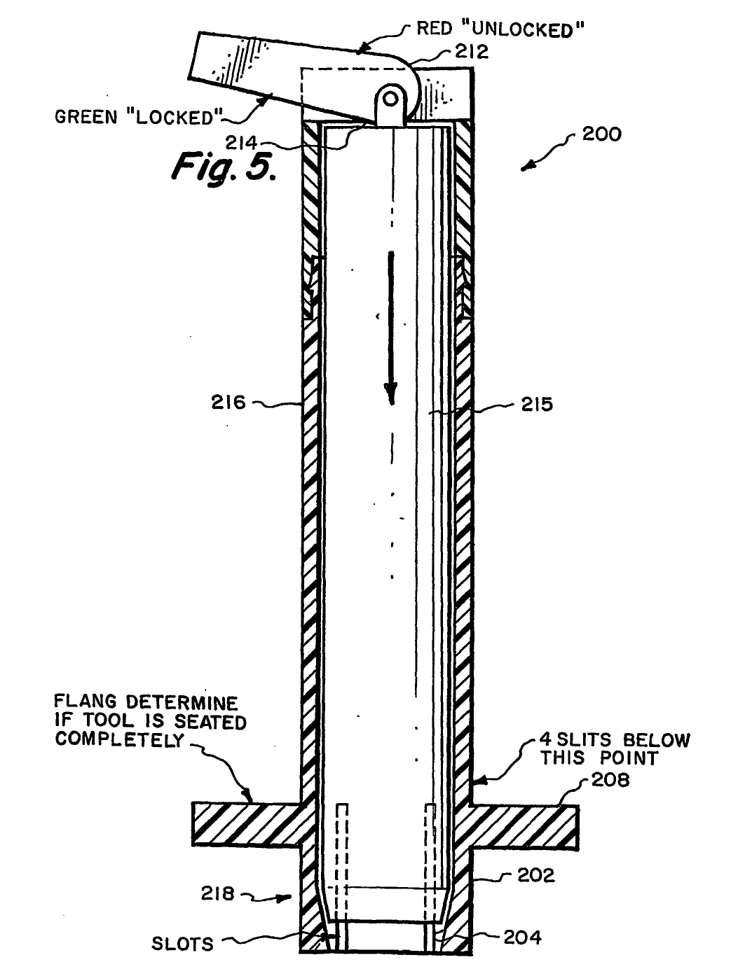

An insertion device 100 with an expandable tip 102 is illustrated in FIGS. 4-5. Referring to FIG. 4 the tip 102 is inserted into the inlet side of the device and expanded against the I.D. of the cylindrical portion of the body. Protrusions 103 on the expanding tip of the insertion device would interlock with a groove 108 in the I.D of the body 14 to provide additional purchase. The foremost portion or head 110 of the insertion device 100 would be non-expanding and locate into the cylindrical distal portion 112 of the cartridge 12 (prior to the cone) during insertion for greater stability. Use of a strapless insertion device simplifies and speeds up the insertion process.

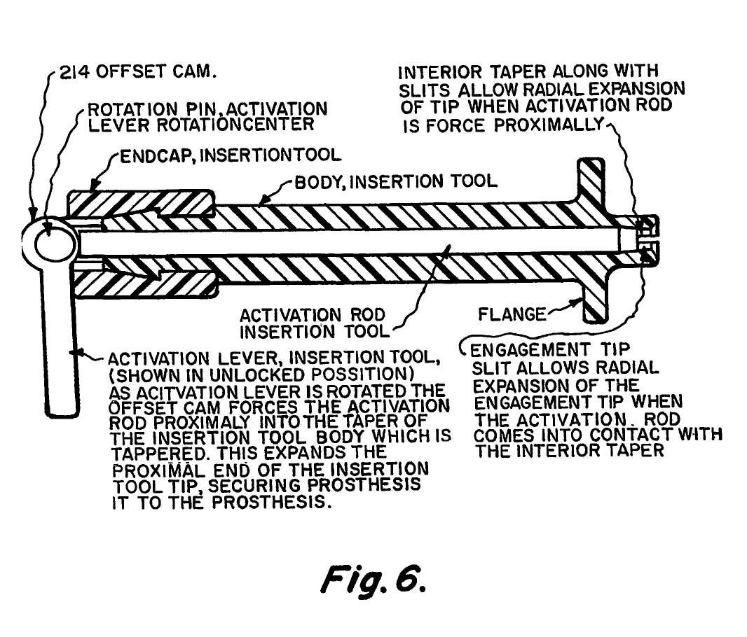

Referring now to FIGS. 5 and 6 an insertion device 200 with radially expandable tip 202 is inserted into the proximal side of the prosthesis 10 and expanded against the interior surface 204 of the cylindrical portion 208 of the prosthesis 10 securing the two together therefore allowing strapless insertion. The expanding tip 202 (distal end) of the insertion tool is placed into the proximal end of the voice prosthesis until the prosthesis contacts an insertion tool stop flange 208. The stop flange 208 is also a visual aid to indicate to the clinician that the insertion tool has been inserted correctly. When the activation lever 212 is rotated from the unlocked to locked position, the cam 214 on the activation lever 212 forces the activation rod 215 distally through the insertion tube 216 into the small end of the taper. This in turn causes the slotted portion 218 of the insertion tool tube 216 to expand radially. The activation lever 212 can be labeled such that from the view of the clinician (during valve insertion), the insertion tool activation lever 212 will read either a green “Locked” or red “Unlocked”. The tip 202 expands into the proximal end of the prosthesis cartridge, locking the insertion tool and voice prosthesis together. At this time the prosthesis can be inserted into the patient. When properly located the activation lever 200 is rotated to the unlocked position, the insertion tool is removed from the prosthesis and patient.

Use of a strapless insertion device simplifies and speeds up the insertion process. The device requires fewer and less complicated steps. A strapless insertion method is not currently used with any available voice prosthesis.

The illustrated insertion device uses a cam to move the actuating rod axially but this could also be accomplished with a:

a. Rack and pinion located on the side of the device. The actuating rod would be the rack and the lever would be the pinion gear.

b. A twisting motion with a spiral ramp could be used to create axial motion.

The insertion tool expands against the inside of a cylinder to secure itself to the prosthesis. Securing the insertion tool to the prosthesis could also be accomplished by having the tip of the insertion tool compress around some portion of the prosthesis.

All components of the Strapless Insertion Tool could be made in a mold from one thermoplastic resin, suitably Delrin or Kynar, or any rigid plastic or metal material.

The mechanical function of the voice prosthesis device is similar to that of a typical shut-off valve. In addition, at the distal portion of the valve there is a debris flap to reduce the amount of particles allowed into the device.

To create a seal, a ball, cone or annular ring such as a ruby ball is preloaded into the cone of the cartridge by a helical stainless steel spring. The spring is held in place by the end cap, which snaps onto the distal end of the cartridge capturing the helical spring between the cartridge and end cap. The end cap also limits the ruby ball from moving distally to a position where damage to the spring occurs. The cartridge body is the interface between the interior components and the esophagus.

The voice prosthesis device of the invention can use unique materials that do not allow microbial colonization of the working components of the device (ruby or metal ball, metal helical spring, Delrin or Kynar cartridge) and therefore will greatly extend the working life of the device. Microbial colonization prevents the currently marketed prosthesis from (over time) forming a complete seal, causing leaks and the need for replacement.

The working parts of the device are made of materials (Ruby, metal, Delrin or Kynar) that will not change their performance characteristics (pressure vs. flow, opening and closing pressures) with regard to time and/or usage. Preload is provided by the stainless steel helical spring and this forms the basis for the performance characteristic of the valve. Devices currently marketed are made from silicone elastomers, which exhibit substantial performance change with time and usage, specifically when preloaded. A metal (preferably stainless or titanium), plastic, or composite spring will offer substantially greater consistency and performance to the patient over the lifetime of the product.

Metals and composite materials are excellent spring materials as compared to elastomers and will yield springs that are consistent in their spring rate from spring to spring and lot to lot. The elastomers used as a spring material on all currently marketed valves vary in their elastic properties from lot to lot as well as produce springs with varying spring rates. A metal/plastic or composite helical spring will provide the patient and clinician with a more consistent product, while reducing the need for in-house testing and will improve yields.

The design of the double or other multiple configuration helical spring allows the spring to compress and extend uniformly, without drifting off axis. This, along with the flat platform at the small end of the spring (which is in contact with the ruby ball), allows the ruby ball to self-center into the cone to allow proper sealing. Wire springs do not allow for this self-centering action and may not allow the ball to seat properly. The cartridge cone also aids in the self-centering of the ruby ball during sealing as well as improving aerodynamics. Other possible design configurations for the spring are triple helix, leaf springs, x-spring; washer type springs (wave, bevel and the like).

Pins in the cartridge body (cartridge retainer pins) fit into notches located in the outer ring of the helical spring. These pins will locate the spring or springs to facilitate faster and more precise location during assembly. The cartridge retainer pins can also be used to stake the spring if ultrasonic welding of the cartridge body to end cap is performed.

The cartridge retainer pins positively locate the spring(s) in place such that only the breaking of the spring or pins would allow the spring to become separated from the device. This provides additional safety to the patient.

The cartridge retainer pins also allow multiple springs to be placed in the device such that their orientation would prevent coil binding and present a similar air path profile (pressure drop) as that of a single spring. Stacking springs will allow a greater spring rate at any given spring travel or preload. This will provide greater flexibility in performance and design of the device.

The pins described above are molded into the cartridge and lock into holes in the outer coils of the helical springs, however, the pins could also be located in the end cap, or the helical spring could be formed with tabs that located in holes molded in the cartridge or end cap.

By having the outer ring of the spring captured within (between) the walls of the cartridge and cartridge end cap, the outer ring of the helical spring is kept out of the air path for improved aerodynamics and therefore better overall performance. This provides greater airflow. This is important to the performance of the voice prosthesis.

The use of Delrin or Kynar in the cartridge and the end cap will permit the use of ultrasonic welding for bonding during assembly instead of adhesives which can be time consuming in their application and inconsistent in performance. Ultrasonic welding can be beneficial as compared to snap fit features, which can add substantial cost to the mold. Other attachment methods to the main cartridge could be via snap press fit or threaded with or without adhesive.

The end cap will also prevent the over extension of the helical spring by preventing the ball from moving so far distally as to cause permanent deformation of the helical spring such as during extreme vocalization (screaming), improper cleaning, or to control the location of the ball during vocalization to provide optimum flow characteristics. This could be accomplished by having a bar crossing the diameter of the end cap thus stopping over excursion of the ruby ball. This would insure the proper long-term function of the spring.

This design of the voice prosthesis of the invention is unique in that the device has identical opening and closing pressures. This is not the case with the currently marketed voice prosthesis and provides more consistent operation for the user.

A silicone or polyurethane flap on the exhaust side of the device can be used to prevent or reduce particles from entering the prosthesis from the esophagus. This flap would not be used as an air seal and would be extremely flexible to limit pressure drop. A non-sealing debris flap of this type is not used in the industry. The flap could be attached with adhesives, or could be a snap fit onto pins molded into the body that would be inserted through holes in the flap. This snap fit could be assisted with an adhesive as needed. This debris flap function could also be performed with a duckbill construction.

For the cartridge and end cap, either of the following materials could be used. Delrin-This material is resistant to microbiological colonization; has a history as an implantable material, can be ultrasonically welded, and is considered to be a very dimensionally stable plastic. Kynar-This material is resistant to microbiological colonization, has a history as an implantable material, can be ultrasonically welded.

Stainless Steel and titanium are excellent materials for the helical spring. The materials are resistant to microbiological colonization and have histories as implantable materials. In comparison to the currently marketed products which use elastomer as springs, steel is vastly more consistent and has an infinite working life. The spring can be formed by chemical milling or etching and then bent into a helix. The cold forming process does not affect the mechanical properties of the metal. The spring could also be formed by stamping.

Ruby is resistant to microbiological colonization and has a history as an implantable material. It can be purchased in very precise spherical diameters inexpensively for use as the ball.

For the body of the prosthesis and particle flap either of the following materials could-be used. Silicone elastomer has a history as an implantable material. Polyurethane resin also has a history as an implantable material and has an added benefit of being resistant to microbial colonization. The resins used for the body and flap suitably have a durometer from 70-80.

It is to be realized that only preferred embodiments of the invention have been described and that numerous substitutions, modifications and alterations are permissible without departing from the spirit and scope of the invention as defined in the following claims.

1. A valve for a voice prosthesis comprising in combination:
a hollow, rigid cartridge having a proximal end and a distal end;
a ball having a first diameter slidingly disposed in the cartridge;
an open spring having a first end mounted at the distal end of the cartridge and has a second end within the cartridge for mounting the ball. The spring biases the ball toward the proximal end of the cartridge and is compressible toward the distal end of the cartridge on application of low pressure to the proximal surface of the ball;
the proximal end of the cartridge having a diameter smaller than the ball and sealingly receiving the ball.

2. A valve according to claim 1 further including an open end cap received on the distal end of the cartridge and containing means for mounting the first end of the spring.

3. A valve according to claim 2 in which the mounting means includes locking pins for locking the first end of the spring.

4. A valve according to claim 1 in which the spring is a helical spring.

5. A valve according to claim 4 in which the helical spring is formed of at lease one spiral of flat material that compresses into a flat configuration.

6. A valve according to claim 5 in which the spring has a double helical configuration.

7. A valve according to claim 1 in which the ball has a smooth surface impervious to microbial growth.

8. A valve according to claim 7 in which the ball is formed of a ceramic material.

9. A valve according to claim 8 in which the proximal portion of the ball has a circular cross-section.

10. A valve according to claim 9 in which the proximal portion of the ball has a spherical or conical shape.

11. A valve according to claim 1 in which the proximal end of the cartridge has a conical shape.

12. A voice prosthesis including in combination;
a tubular, hollow, elastomeric body having a central channel terminating in a proximal end and a distal end, the valve as defined in claim 1 being fixedly received in the channel.

13. A voice prosthesis according to claim 12 further including a particle flap hingedly mounted at the distal end of the body.

14. A voice prosthesis according to claim 12 in which the proximal end of the body contains a hood extending axially from the proximal end of the cartridge.

15. A voice prosthesis according to claim 14 in which the hood contains a groove for locking engagement with the end of an insertion device.

16. A voice prosthesis insertion device for inserting the voice prosthesis as defined in claim 12 into a trachea-esophageal fistula comprising in combination:
an elongated handle to be gripped by the hand of the user, the handle having a proximal end and a distal end;
the handle having a distal tip having a diameter such that it is slidingly received into the proximal end of the rigid cartridge;
said handle including means for expanding the distal end of the insertion tool to lock the distal end in place in the cartridge; and
activation means on said handle for activating the expansion means.

17. A device according to claim 16 in which the distal end of the handle is slotted.

18. A device according to claim 16 in which the distal end of the handle contains a protrusion which on expansion of the distal end engages and locks into the groove as defined in claim 15.
